(19) United States
(12) Patent Application Publication
(10) Pub. No.: US 2008/0050355 A1
(43) Pub. Date: Feb. 28, 2008

(54) PROTECTION OF BIOACTIVE FOOD INGREDIENTS BY MEANS OF ENCAPSULATION

(75) Inventor: Sophie VASLIN, Saint Cloud (FR)

Correspondence Address: FOLEY AND LARDNER LLP;SUITE 500, 3000 K STREET NW, WASHINGTON, DC, 20007, US

(73) Assignee: COMPAGNIE GERVAIS DANONE, Levallois-Perret (FR)

(21) Appl. No.: 11/577,617

(22) PCT Filed: Oct. 21, 2005
(86) PCT No.: PCT/EP05/55440

§ 371 Date: Apr. 20, 2007

(30) Foreign Application Priority Data
Oct. 22, 2004 (FR) 0411273

(51) Int. Cl. A61K 35/74

(52) U.S. Cl. 424/93.45

(57) ABSTRACT

The invention relates to a food product containing one or more live micro-organisms and at least one bioactive food ingredient of interest, in which the bioactive food ingredient(s) of interest is protected by means of encapsulation in a fat, such as to reduce the metabolisation thereof by the aforementioned live micro-organisms.

The present invention relates to a food product containing one or more living microorganisms and at least one bioactive food ingredient of interest, in which the living microorganisms) and said bioactive food ingredients) of interest are used in a manner that reduces the metabolisation of said bioactive ingredients) by said microorganism(s).

The market for bioactive or functional food ingredients, peptides in particular (i.e. having beneficial action for the consumer either locally in the digestive tube, or a remote effect in the body after passing through the circulatory system) has been fast-expanding in recent years.

Bioactive peptides are defined sequences of amino acids which are inactive in their protein of origin, but which have special properties once released by enzymatic action. They are also called functional peptides. These bioactive peptides are able to have an effect inter alia on the digestive system, the body's defenses (e.g. an antimicrobial or immunomodulator effect), the cardiovascular system (in particular an antithrombotic or antihypertensive effect), and/or the nervous system (such as a sedative, analgesic effect of opioid type) (see tables 1 and 2 below).

Table 1 below lists the main functional peptides released by hydrolysis of the proteins of human milk and cow's milk.

TABLE 1

| | Functional | Milk | | |
|---|---|---|---|---|
| Original proteins | peptides | origin** | | Described activities |
| α-casein | α-casomorphin | | C | opioid |
| | casein | | | |
| | α-exorphin | | C | opioid |
| | casokinin | | C | antihypertensive |
| β-casein | β-casomorphin | H | C | opioid |
| | casokinin | H | C | immunomodulator + |
| | CPP | H | C | antihypertensive |
| | | | | action on minerals |
| κ-casein | CMP = GMP | | C | modulated gastro- |
| | | | | intestine motility & |
| | | | | release of digestive |
| | | | | hormones |
| | casoxin | H | C | opioid antagonist |
| | casoplatelins | | | antithrombotic |
| α-lactoalbumin | fragments 50-53 | H | C | opioid |
| β-lactoglobulin | β-lactorphins | | C | opiate + anti- |
| | | | | hypertensive |
| lactoferrin | lactoferroxin | H | C | opioid antagonist |
| lactotransferrin | | | | |

(*) the amino acid sequences are not exactly the same
**H: human milk/C: cow's milk

Table 2 below summarizes the main physiological activities of milk-derived functional peptides known at the present time.

TABLE 2

| Activity | Peptides | In vitro | In vivo animal | In vivo man | Reference |
|---|---|---|---|---|---|
| Effect on digestion | Caseinomacro- | CCK production by rat intestine cell | | | Beucher 1994 |
| | peptide (CMP) | | | | |
| | | | Calf: after ingesting CMP | Man: after | Yvon 1994 |
| | | | (210 mg/kg), inhibition of gastric | ingesting | |
| | | | secretion and reduced CKK plasma | CMP(4 g) reduced | |
| | | | concentration | acid secretion | |
| | β-casomorphins | | Rabbit: after placing in lumen, | | Ben Mansour |
| | | | anti-secretory effect on the ileum | | 1988 |
| | | | Dog: after intragastric | | Schusdziarra |
| | | | administering, modulation of post- | | 1983 |
| | | | prandial insulinaemia; | | |
| | | | cancellation of this effect by | | |
| | | | naloxone | | |
| | natural β- | Several effects at rabbit ileum | | | Tomé 1987 1988- |
| | casomorphins and | | | | Mahé 1989 |
| | some of their | | | | |
| | analogs | | | | |
| | Non-metabolised | Stimulated intestinal absorption of electrolytes | | | Ben Mansour |
| | analogs of β- | | | | 1988 |
| | casomorphins | | | | |
| | Casein | | Dog: 10 g casein/300 mL water | | Defilippi 1986 |
| | | | administered by stomach tube: | | |
| | | | inhibition of small intestine | | |
| | | | motility cancelled by naloxone. | | |
| | | | vs. 10 g soy protein: no effect | | |
| Anti-microbial effect | Lactoferricin | Inhibited growth of pathogenic strains | | | Tomita 1994-Zucht |
| | Casocidin 1 (α-S₁₋ | | | | 1995 |
| | casein)165-203 | | | | |
| | α-S₁B-casein fragment | Inhibited growth of pathogenic | Mouse, Sheep: effective by | | Lahov 1996 |
| | (1-23 N terminal) = isracidin | strains | IM injection against | | |
| | | | Stapylococcus aureus | | |
| | Human β-casein fragment | | Mouse: protective effect by | | Migliore- |
| | | | IV injection against | | Samour 1989 |
| | | | K. pneumoniae | | |
| Immunomodulator effect | Fragments of bovine α- | Proliferation of human | | | Kayser 1996 |
| | lactalbumin and bovine | lymphocytes (PBLs) activities | | | |
| | κ-casein | via Con A | | | |
| | Synthetic β-casokinin 10 | Proliferation or suppression | | | Kayser 1996 |
| | and β-casomorphin 7 | of PBLs depending on | | | |
| | | concentration | | | |
| | Human β-casein 54-59, | Stimulated phago-cytosis of | | | Parker 1984 |
| | α-lactalbumin 51-53 | sheep red cells by mouse | | | |
| | | peritoneal macrophages | | | |
| | Bovine β-casein | Stimulation of mouse | No in vivo protection | | Migliore- |
| | Casein 191-193 | peritoneal macrophages | | | Samour 1988 |
| | Casein 63-68 | | | | |
| | Bovine κ-casein | Inhibited B-lym-phocyte | | | Otani 1992 |
| | Caseinomacro- | prolifer-ation of Peyer's | | | 1995 |
| | peptides(106-169) | patches in mouse and rabbit | | | |
| Antithrombotic effect | Bovine caseino- | | | CGP isolated in newborn | Chabance |
| | glycopeptide(bCGP) | | | plasma after ingesting | 1995 |
| | Human caseinogly-copeptide | | | formula or mother's milk | |
| | (hCGP) | | | | |
| | Peptide 106-116 of bovine κ- | Inhibits | | | Jollès |
| | casein | platelet | | | 1986 |
| | | aggregation | | | |
| | Human lacto-transferrin tetra-peptide (39-42) | Inhibits | | | Raha 1988 |
| | | platelet | | | |
| | | aggregation | | | |
| | | | Rat/guinea-pig with test | | Drouet |
| | | | arterial thrombosis: after IV | | 1990 |
| | | | injection antithrombotic | | |
| | | | activity | | |
| Antihypertensive effect | Enzymatic hydrolysates of β- | Inhibition of | | | Mullaly |
| | lactoglobulin and α-lactalbumin | ACE | | | 1997 |
| | Synth. fragments of human β- | Inhibition of | Rats given angiotensin 1: | | Kohmura |
| | casein | ACE | after IV injection return to | | 1989 |
| | | | initial blood pressure | | |
| | Milk peptides fermented with | | Hypertensive rats: ingestion | | Masuda |
| | L. helveticus and S. cerevisiae | | 10 mL fermented milk/kg body | | 1996 |
| | | | wt: peptides found in the | | |
| | | | aorta with ACE inhibition. | | |
| | Peptides from milk fermented | | Hypertensive rats: after | | Yamamoto |
| | with L. helveticus | | ingestion, reduced blood | | 1994 |
| | | | pressure. | | |
| | Peptides from milk fermented | | Hypertensive rats: after | | Nakamura |
| | with L. helveticus + S. cerevisiae | | ingestion, reduced blood | | 1995 |
| | Val-Pro-Pro | | pressure. | | |
| | (VPP)/Il-Pro-Pro (IPP) | | Normal rats: no effect | | |
| | | | | Human hypertension (36 | Hata 1996 |
| | | | | patients): after 8 weeks' | |
| | | | | ingestion of 95 mL/d, | |
| | | | | reduced blood pressure | |
| Opioid effects | β-casomorphins | | Rats: after intra-carotid | | Ermisch 1983 |
| | | | injection accumulation of β- | | |
| | | | casomorphins in area with | | |
| | | | no blood brain barrier. | | |
| | | | Newborn calves: after first | | Umbach 1985 |
| | | | meal of cow milk β- | | |
| | | | casomorphins in the blood | | |
| | | | Piglets: after ingesting | | Meisel 1986 |
| | | | bovine casein, β-casomorphin | | |
| | | | isolated from duodenal | | |
| | | | chyme. | | |
| | | | Puppies: after ingesting | | Singh 1989 |
| | | | mother's milk, β- | | |
| | | | casomorphins found in the | | |
| | | | blood | | |
| | | | | Man: after ingesting | Svedberg 1985 |
| | | | | cow's milk, β- | |
| | | | | casomorphins found in | |
| | | | | content of small | |
| | | | | intestine, | |
| | | | | but not in adult blood. | Teschemacher |
| | | | | | 1986 |
| | Peptides of | Opioid effect on | | | Yoshikawa 1986 |
| | synthetic human | isolated ileum of | | | |
| | β-casein | guinea-pig, | | | |
| | | cancelled by | | | |
| | | naloxone | | | |
| | Bovine and human | Antagonist opioid | | | Chiba 1989 |
| | casoxins | effects on isolated | | | |
| | (κ-casein) | ileum muscle of | | | |
| | | guinea-pig. | | | |

These peptides are most often obtained by hydrolysis of vegetable proteins (e.g. soy proteins) or animal proteins (e.g. caseins or milk serum proteins), hydrolysis being generated by enzymatic and/or fermenting processes, most often accompanied by concentration of the active fraction, a step that is generally necessary to provide the targeted “health benefit”. The fabrication and use of these peptides for a health benefit are the subject of an abundant literature (see in particular Danone World Newsletter N°17, September 1998).

Among the food vectors likely to receive said ingredients, fermented milk products rank in high position through their health benefit due to the presence of ferments and fermentation products (i.e. molecules derived from transformation of the substrates present in the milk by lacetic bacteria). Up until now, the scientific community has given special consideration to the properties of ferments. Researchers have recently taken an interest in fermentation products, among which some peptides occupy a special position since they form numerous specific biological messengers Fermented milk products therefore appear to be particularly suitable as vectors for hydrolysates of bioactive peptides obtained from milk substrates for example, such as caseins or serum proteins.

One major problem arises in this case: the microorganisms, and in particular the lacetic bacteria used for the production of fresh milk products (e.g. yoghurts, fermented milk preparations, milk-based fermented beverages, etc.) are generally capable of consuming peptides to meet their nutritional needs and more particularly their nitrogen requirements. In this respect reference will later be made to the “metabolisation of peptides”. Lacetic bacteria effectively have several degradation and/or transport systems enabling them to metabolise peptides and causing them to disappear from the medium:
1/a proteolytic system (wall proteases, PRT) which divides the proteins and large peptides to facilitate their uptake (“extracellular metabolisation system”).
2/transport systems towards inside the cell, of which one whose size is close to 10 amino acids is specific to oligopeptides, the other being adapted for the transport of di- and tripeptides (lactobacilli have an additional tri-peptide permease system) (“transport system(s) towards inside the cell”) and
3/An intracellular enzymatic system able to degrade the peptides into amino acids (comprising around fifteen endo- and exopeptidases (“intracellular metabolisation system”).

Since the quantity of peptides naturally present in milk is generally too low for the needs of lacetic bacteria, it is usual to accelerate their growth by providing additional peptides. These are fully consumed during fermentation.

To conclude, owing to: (1) the nitrogen needs of lacetic bacteria for which peptides form the main source in milk, (ii) the capacity of these bacteria to consume peptides efficiently, and (iii) the survival of a large population of lacetic bacteria in milk-based fermented products, up until the Best Before Date (BBD), the use of ingredients containing functional peptides for the production of fermented milk products is difficult and even impossible, since these ingredients are most often consumed by the lacetic bacteria during fermentation and even during storage of the products up until the BBD.

In addition, not only is this problem of degradation of the peptides through “undue” metabolisation by bacteria non-specific to a given peptide, but it is not specific either to a particular ferment (or microorganism, preferably a bacterium, capable of fermenting).

This is a general problem which arises irrespective of the peptide(s) or microorganism(s) under consideration.

By way of example, mention may be made of the bioactive peptide αS1[91-100] (see European patent EP 0 714 910: a peptide with relaxing properties contained in the hydrolysate of milk proteins, marketed in particular by Ingredia: 51-53 Avenue Fernand Lobbedez BP 946 62033 ARRAS Cedex, France, under the name Lactium®). The Applicant has observed that the population of living lacetic bacteria in the end product continues to metabolise the bioactive peptide during storage of the end product, so that after only 10 days (for fresh products with a BBD of 28 days) between 35 and 55% of the αS1[91-100] peptide has disappeared, which is fully unacceptable to guarantee a “health” effect for consumers (data not shown).

Since consumption of the bioactive peptide is due to the metabolic activity of the ferments, it could be contemplated to reduce this phenomenon by destroying all or part of the microorganisms, e.g. using suitable heat treatment (thermisation or pasteurisation). In this case, it is possible to preserve the αS1[91-100] peptide (e.g. after heating to 75° C. for around 1 min).

However said solution has numerous drawbacks:
thermisation of a fermented milk mass implies the use of stabilisers added before the heat treatment (pectins, starches, carrageenans etc.) which complicates the process and substantially increases the cost of the formula;
the industrial production line is more complex and requires specific, higher investment;
the product no longer benefits from the quality labels for products containing living ferments (of yoghurt type) and thereby loses the benefits associated with the consumption of lacetic ferments; and
the organoleptic impact, generally negative, is significant.

There is therefore a need for a food product containing both living microorganisms e.g. a yoghurt, and one or more bioactive food ingredients of interest, in which these bioactive food ingredients of interest are protected against metabolisation by said living microorganisms, whilst preserving the organoleptic qualities of the food product.

Under the present invention, the Applicant provides a solution which can meet this existing need.

The present invention therefore focuses on a food product containing one or more living microorganisms and at least one bioactive food ingredient of interest, characterized in that said living microorganism(s) and said bioactive food ingredient(s) of interest are used in a manner which reduces the metabolisation of said bioactive ingredient(s) by said living microorganism(s).

Therefore the Applicant has been able to show that one or more bioactive food ingredients of interest can be efficiently protected against metabolisation by living microorganisms, provided that suitable conditions are applied for their combined use.

Said suitable implementing conditions may have recourse to various means, among which:

a) the use of living microorganisms whose capacity to metabolise bioactive ingredients is reduced; and/or

b) the use of decoy food ingredients which are deliberately “given as fodder” to the living microorganisms; and/or

c) the use of a physical protection for the bioactive ingredients, in particular through their encapsulation.

It will be noted in this respect that one or more, and even all these means, may advantageously be combined within one same food product.

The Applicant therefore proposes a food product containing one or more living microorganisms and at least one bioactive food ingredient of interest, characterized in that said bioactive food ingredient(s) of interest are protected:
physically, said bioactive food ingredient(s) of interest preferably being encapsulated; and/or
by means of at least one decoy food ingredient contained in said food product, so that their metabolisation by said living microroganism(s) is reduced.

More precisely, one subject-matter of the present invention is a food product containing one or more living microorganisms and at least one bioactive food ingredient of interest, characterized in that said bioactive food ingredient(s) of interest are physically protected by encapsulation in a fat, so that their metabolisation by said living microorganism(s) is reduced.

As briefly indicated in the preceding general description, by “metabolised” or “metabolisation” under the present invention is meant the transformation or degradation of a substance by one or more living microorganisms, for consumption as nutritional source, and whose end consequence is its more or less complete disappearance from the medium.

In the meaning of the invention, the metabolisation of an ingredient is “reduced” if it is lower than the metabolisation of the same ingredient when the latter is not protected by at least one of the means provided by the present invention.

Advantageously, and ideally, this reduced metabolisation tends towards or is even zero, which amounts to little, almost none and even no metabolisation of said ingredient.

According to one particular embodiment of the present invention, the residual quantity of bioactive food ingredient(s) of interest in said food product, 3 weeks after its preparation, lies between approximately 50 and 100% compared with the quantity of bioactive food ingredient(s) of interest present in the product just after its preparation.

Preferably said residual quantity lies between approximately 80 and 1000.

By “residual quantity of bioactive food ingredient(s) of interest in said food product” according to the present invention is meant the percentage of bioactive food ingredient(s) of interest present in said food product when this product is stored under suitable storage conditions (e.g. in the order of 4 to 10° C. for a fresh product) for 3 weeks, as compared with the percentage of bioactive food ingredient(s) of interest initially present i.e. just after the manufacture of the product.

When implementing the present invention, the bioactive food ingredients(s) of interest are chosen in particular from among:
proteins
peptides
vitamins
micronutrients
derivatives or analogs thereof, and
their combinations
   Preferably the bioactive food ingredient of interest is chosen from among:
proteins
peptides
analogs or derivatives thereof, and
their combinations.

Preferably the bioactive food ingredient of interest is chosen from among: peptide αS1[91-100] (cf. European patent 0714910), peptide C6-αₛ₁194-199 (cf. U.S. Pat. No. 6,514,941), peptide C7-β177-183 (cf. U.S. Pat. No. 6,514,941), peptide C12 αₛ₁23-34 (cf. U.S. Pat. No. 6,514,941), caseinophosphopeptides, α-casomorphin, α-casein exorphin, casokinin, β-casomorphin, Caseino-MacroPeptides (CMPs) also called GlycoMacroPeptides (GMPs) or CaseinoGlycoMacroPeptides (CGMPs), casoxin, casoplatelins, fragments 50-53, β-lactorphins, lactoferroxin, the peptides Val-Pro-Pro (cf. European patent EP 0583074), Lys-Val-Leu-Pro-Val-Pro-Gln (cf. application EP 0737690), Tyr-Lys-Val-Pro-Gln-Leu (cf. patent EP 0737690), Tyr-Pro (cf. application EP 1302207 and patent EP 0821968), Ile-Pro-Pro (cf. Nakamura et al. 1995; and Japanese patent JP 6 197 786), fragments, analogs and derivatives thereof, proteins and/or protein-containing peptides and their combinations (for a review see in particular Danone World Newsletter N°17, September 1998).

Further preferably, the bioactive food ingredient of interest is chosen from among: peptide αS1[91-100], fragments, analogs or derivatives thereof, proteins and/or protein-containing peptides, and their combinations.

By “analog” is meant any modified version of an initial compound, here a protein or a peptide, said modified version possibly being natural or synthetic, in which one or more atoms such as carbon, hydrogen oxygen atoms, or heteroatoms such as nitrogen, sulphur or halogen have been added to or deleted from the structure of the initial compound, so as to obtain a novel molecular compound.

A “derivative” in the meaning of the invention is any compound which has a resemblance or structural pattern in common with a reference compound (protein or peptide). This definition also covers firstly compounds which, either alone or with other compounds, can be precursors or intermediate products in the synthesis of a reference compound subject to one or more chemical reactions, and secondly compounds which can be formed from said reference compound, either alone or with other compounds, via one or more chemical reactions.

The above definition of “derivatives” therefore at least covers the hydrolysates, trypsic in particular, of proteins and/or peptides, fractions of hydrolysates and mixtures of hydrolysates and/or of hydrolysate fractions.

Also the above terms “analog” and “derivative of a peptide or protein” cover a peptide or protein that is glycosylated or phosphorylated, or which has undergone any chemical group grafting.

In another embodiment of the present invention, the bioactive food ingredient of interest may in particular be a sugar or a fatty acid.

Under the present invention, only said bioactive food ingredient(s) of interest are encapsulated; the living microorganisms are not encapsulated.

By “encapsulated” or “encapsulation” according to the present invention is meant the use of a method to protect an active ingredient in a vehicle of microparticle type, to permit controlled release of this active ingredient. In the case in hand, the active ingredient consists of one or more bioactive food ingredients of interest.

In highly advantageous manner, encapsulation can remedy the disadvantages of prior art solutions, in that it prevents the metabolisation of the bioactive food ingredients of interest by the living microorganisms.

Additionally, encapsulation enables the bioactive food ingredients of interest to travel as far as the intestine without being degraded, and to pass through the intestinal barrier without damage so that they can produce their effects therein.

Also, the Applicant points out that encapsulation of substances is fully original with respect to fermented food products, in particular fermented milk products.

Finally, it is to be stressed that in fully interesting manner, encapsulation makes it possible to obtain an end product that is organoleptically more acceptable e.g. by masking the greater or lesser bitterness of some bioactive ingredients, in particular of some peptides.

This is why, according to one particularly preferred embodiment, the present invention concerns a food product such as previously described whose bitterness is reduced.

In the meaning of the invention, the bitterness of a food is <<reduced” if a food is considered as less bitter after a pairing test [Directional Paired Comparison Method, Sensory Evaluation of Food, Harry T. Lawless, Holdegarde Heymann (1990)]. This test can be conducted by a panel (of 10 or more members) grouping together different persons who have fully integrated the notion of bitterness (learning of this notion is achieved by tasting products containing a bitter molecule (e.g. caffeine) these products being made more or less bitter according to the concentration of said molecule). The product conforming to the present invention is then subjected to blind testing (the panel members do not know which product is first presented) and is compared by these panel members with a product not conforming to the invention. Evidently, the two products presented to the panel only differ in that one is produced according to the invention and not the other. The order of presentation of the two products from one panel member to another is random, the number of persons receiving firstly the product conforming to the invention being equal to the number of persons receiving the other product first. Each member must indicate, on each repeat of the test, which is the bitterest product of the two products tasted.

Under the invention, said bioactive food ingredients of interest are encapsulated in a fat.

Fat encapsulation can be conducted in particular by dispersing the bioactive food ingredient(s) of interest in a fatty phase in which they are finally encapsulated (i.e. imprisoned, trapped inside very fine lipid droplets).

It will be noted that, unlike known dispersion methods particularly used in other technical areas such as cosmetology, the use of said dispersion does not require any subsequent drying step.

According to usual acceptance, by “fat” or <<fatty body” is meant any substance containing one or more lipids. It may for example be an oil, a fat, butter, etc. This fat may be natural, therefore existing in diverse forms in animals, vegetables, and in their products (i.e. products derived from their metabolism) or synthetic.

One selection criterion for the fat which can be used for the present invention is related to its melting point. To achieve dispersion as envisaged above, a concrete fat must be used i.e. solid at room temperature. Among suitable oils particular mention may be made of palm oil and its fractions, coconut oil and its fractions, palmist oil and its fractions, vegetable butter oils of cocoa type, margarines, hydrogenated or partly hydrogenated vegetable oils, and analogs.

The choice of concrete fat is also made taking into account its nutritional qualities. In this respect preference is given for example to fractionated fats rather than hydrogenated or partly hydrogenated fats.

If it is desired to disperse more efficiently, even to solubilise the bioactive food ingredient, encapsulation is preferably implemented in a multiple water/oil/water emulsion. In this case, the use of fats that are fluid at room temperature (oils) may be more suitable (rapeseed, oleic rapeseed, soy, sunflower, oleic sunflower, fish oils, algae oils, etc.).

In this respect, the Applicant points out that through the choice of a concrete fat, use can be made of its recrystallization after melting, leading to the trapping and physical protection of the bioactive food ingredient.

A suitable fat may in particular be chosen from among animal fats, in particular milk or fish fats, and vegetable fats.

It is specified that fish extracted fats are of particular interest for their high content of polyunsaturated Omega 3 fatty acids.

Among the suitable vegetable fats, palm oil, rapeseed oil and/or an alga-extracted fat can be given particular choice.

According to one particular embodiment of the present invention, said living microorganism(s) have an intact or reduced capacity to metabolise said bioactive food ingredient(s) of interest.

According to the present invention, a “reduced metabolisation capacity” is such that the quantity of bioactive food ingredients of interest metabolised during fermentation (which therefore disappear from the medium) is 40% or less of the initial quantity of ingredients (before fermentation).

This translates mathematically as:
Qᵣ≧0.6Qₒ (1)

Wherein:
Qᵣ: residual quantity of bioactive ingredients (present in the medium after fermentation)
Qₒ: initial quantity of bioactive ingredients.

The residual quantity of bioactive ingredients Qᵣ can be measured using a high pressure liquid chromatography method (HPLC) coupled to a detector of MS/MS type. An example of experimental procedure is given in the examples below.

Said living microorganism(s) are preferably bacteria, further preferably living lacetic bacteria.

The living bacteria are more particularly chosen from among:
Streptococcus spp, preferably Streptococcus thermophilus;
Lactobacillus spp;
Lactococcus spp;
Bifidobacterium spp.

Preferably the living bacteria are chosen from among:
Streptococcus thermophilus, deposited with the CNCM collection (Collection Nationale de Cultures des Microorganismes—Institut Pasteur, Paris, France) on 24 Jan. 2002 under number 1-2774;
Streptococcus thermophilus deposited with the CNCM on 24 Oct. 1995 under number 1-1630;
Streptococcus thermophilus deposited with the CNCM on 10 May 2004 under number 1-3211.
Streptococcus thermophilus deposited with the CNCM on 16 Sep. 2004 under number 1-3301; and
Streptococcus thermophilus deposited with the CNCM on 16 Sep. 2004 under number 1-3302.

Further preferably, said living bacteria are S. thermophilus bacteria deposited with the CNCM collection on 10 May 2004 under number 1-3211.

Advantageously, the food product contains at least the living bacteria S. thermophilus and Lactobacillus spp.

Preferably, said living Streptococcus thermophilus bacteria are chosen from among S. thermophilus deposited with the CNCM on 24 Jan. 2 under number 1-2774, S. thermophilus deposited with the CNCM on 24 Oct. 1995 under number 1-1630, S. thermophilus deposited with the CNCM on 10 May 4 under number 1-3211, S. thermophilus deposited with the CNCM on 16 Sep. 2004 under number 1-3301, and S. thermophilus deposited with the CNCM on 16 Sep. 2004 under number 1-3302.

The living microorganism content of the food product according to the invention may vary, and will be chosen by persons skilled in the art in the light of their general knowledge in this area. In practice, a standard global content is sought e.g. in the order of 10⁷ to 10⁹ bacteria per gram of food product.

According to one particular embodiment, a food product conforming to the invention also contains at least one decoy food ingredient.

By “decoy food ingredient” under the present invention is meant a food ingredient (preferably a peptide, a protein, analog or derivative thereof and their combinations) able to act as nutrient source (in particular a nitrogen source) for living microorganisms and intended to be preferably metabolised by said microorganisms, so as to divert these microorganisms away from the bioactive ingredients of interest which are evidently to be given priority safeguarding. Therefore the decoy ingredient is a nutrient source for the microorganisms, which is deliberately sacrificed in order to protect the bioactive ingredients of interest as much as possible. The decoy food ingredient in this respect acts as competing inhibitor of the transport of the bioactive ingredients of interest.

By “food product” here is meant a product intended for animal and/or human consumption, preferably human. This product may be in any type of consumable form. It may be a drink such as water, plant juices (fruit and/or vegetable and/or cereal . . . ) milk, drinkable yoghurts, mixtures therefore. It may also be a solid product, or a more or less moist intermediate product.

According to one particular embodiment, the food product conforming to the invention is a water.

Preferably, the food product according to the present invention is a fermented product.

Further preferably the fermented food product is a dairy or plant product.

By “dairy product” under the present invention, in addition to milk, is meant milk-derived products such as cream, ice-cream, butter, cheese yoghurt; secondary products such as whey, casein; and any prepared product containing milk or milk constituents as main ingredient.

By “plant product” is meant inter alia products obtained from a plant base such as fruit juices, vegetable juices among which soy juice, oat juice or rice juice.

Also the above definitions of “dairy product” and “plant product” each cover any product containing a mixture of dairy and plant products such as a mixture of milk and fruit juice for example.

A further subject of the present invention is a method for encapsulating at least one bioactive food ingredient of interest in a fat.

In this respect, insofar as the addition of a hioactive food ingredient to a melted, concrete fat leads to accelerated crystallisation kinetics thereof, it is important that the fat should be fully melted before adding the ingredient. Therefore the gradual adding of an ingredient under stirring to an aqueous phase itself being stirred allows for slow, induced crystallisation around the ingredient and the formation of oil droplets of adequate size whose distribution is homogeneous.

To add the bioactive food ingredient to a white, preferably fermented, mass it is preferable to prepare an intermediate aqueous medium of syrup type. This syrup is then added to the white mass. The addition of the bioactive food ingredient to the aqueous medium must be made under certain conditions:
(i) total recrystallisation of the fat must be avoided before mixing the bioactive food ingredient and the aqueous medium, in order to ensure gradual, homogeneous adding;
(ii) the temperature of the aqueous medium must be close to the melting point of the encapsulating fat, so as to avoid its recrystallisation when in contact with the aqueous medium; and
(iii) to ensure homogeneous mixing of the bioactive food ingredient and syrup, vigorous stirring (e.g. using a mixer of Ultraturax type) is needed: this gives a white “emulsion” having the texture of a thick cream; also the light consistency of this system is easy to pump when the temperature remains in the region of the melting point of the encapsulating fat.

The use of a food additive such as an emulsifier during encapsulation of the bioactive food ingredient makes it possible to obtain a more homogeneous “population” of fat globules. If no emulsifier is used, it was observed that there is a majority of large fat globules (diameter of around 25 μm and over) and relatively low dispersion of the peptide in the product. On the other hand, when an emulsifier is added, dispersion is higher and the globules are smaller (a maximum diameter of approximately 10 μm).

According to the present invention, the encapsulating method comprises at least:

a) the preparation of a water/fat mixture to be held at a temperature close to the melting point of said fat, preferably within a range of ±10° around said melting point;

b) the gradual incorporation under stirring, preferably gentle stirring, of the bioactive ingredient of interest in the mixture obtained at step a), maintaining the whole at a temperature close to the melting point of said fat, and preferably within a range of ±10° around said melting point; and

c) optionally, the addition of one or more food additives such as emulsifiers, thickeners, etc . . . , and mixing, preferably vigorously mixing, the whole while maintaining the temperature close to the melting point of said fat, and preferably within a range of ±100 around said melting point.

The temperature applied during steps a), b) and optionally c) above may vary slightly from one step to another, while preferably remaining within the range of ±10° around the melting point of the fat. In practice, the temperature applied for all these steps is advantageously substantially the same.

Persons skilled in the art may, if necessary adapt this method in relation to the fat used, using their general knowledge and optionally by conducting simple routine experiments.

Then, to prepare a food product conforming to the invention such as a dairy product, the mixture obtained kept at a temperature close to the melting point of the fat, is incorporated in fully conventional manner into a white mass particularly using pump systems, and preferably after the heat treatment and fermentation steps of this mass.

A further subject of the present invention is a method for preparing a food product such as described above, in which one or more bioactive food ingredients of interest are encapsulated, e.g. according to the above-described method, before being added to the mixture intended to form said food product.

According to one particular embodiment of the present invention, said encapsulated bioactive food ingredient(s) are added to said mixture under agitation.

When the food product is fermented, said encapsulated bioactive food ingredient(s) of interest may be added to the mixture before or after fermentation.

It is preferred however to add the encapsulated bioactive ingredients after fermentation, to preserve the integrity of the fat particles as much as possible against any optional heat treatment steps.

According to one embodiment, the preparation method conforming to the present invention is such that said living microorganism(s) and said encapsulated bioactive food ingredient(s) of interest are added one after the other in the mixture intended to form said food product.

Alternately, the living microorganisms and encapsulated bioactive food ingredients of interest are added simultaneously to the mixture intended to form said food product.

The culture conditions for the microorganisms depend upon said microorganisms and are known to those skilled in the art. By way of example, it is specified that the optimal growth temperatures for S. thermophilus are generally in the region of approximately 36 and 42° C.; they lie between 42° and 46° C. for L. delbrueckii spp. bulgaricus (typically found in yoghurts).

As a general rule, halting of fermentation which depends upon the pH it is desired to achieve, is obtained by rapid cooling, making it possible to slow down the metabolic activity of the microorganisms.

A further subject of the present invention is the use of a food product such as described above as functional food.

By “functional food” is meant a food product which advantageously affects one or more target functions of the body, independently of its nutritional effects. It may for example lead to an improvement in state of health and/or well-being and/or a reduction in the risk of onset of diseases in consumers who ingest normal quantities of said product. As examples of activities of a “functional food”, particular mention may be made of anticancer, immunostimulator, bone health promoting, anti stress, opioid, antihypertensive activities, improved calcium bioavailability, or antimicrobial activity (Functional Food Science in Europe 1998).

Said functional foods may be intended for man and/or animals.

A further subject of the present invention is the use of a fat to encapsulate at least one bioactive food ingredient of interest, intended to be incorporated in a food product containing one or more living microorganisms.

The present invention is illustrated, but not limited by, the following figures:

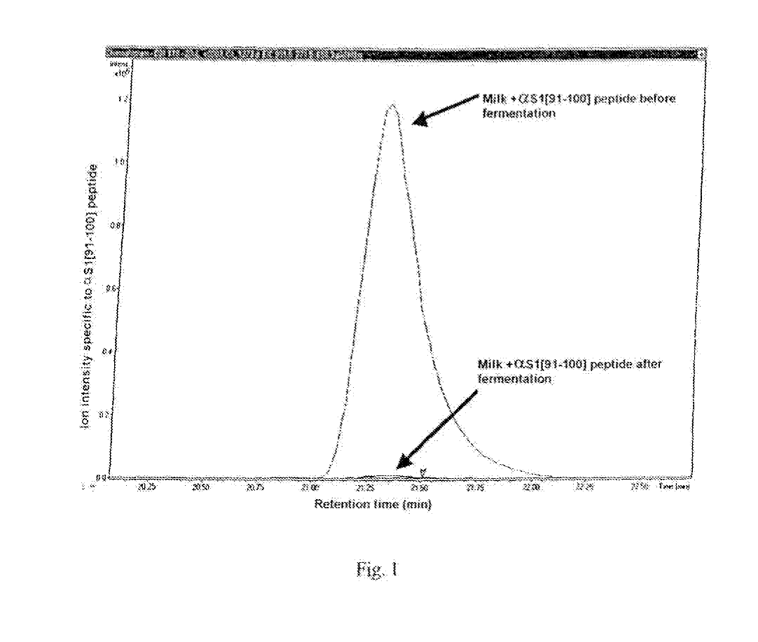

FIG. 1: LC-MS chromatogram illustrating the disappearance of the bioactive peptide αS_{1[}91-100] included in the Lactium® ingredient, during lacetic fermentation. The MS/MS detector is adjusted so as only to show the signal of ions with m/z=634.5 Da (mass of double charged αS_{1[}91-100] peptide) which, after fragmenting, has daughter ions of m/z=991.5 Da; 771.5 Da; 658.3 Da (fragments characteristic of the αS1 [91-100]peptide).

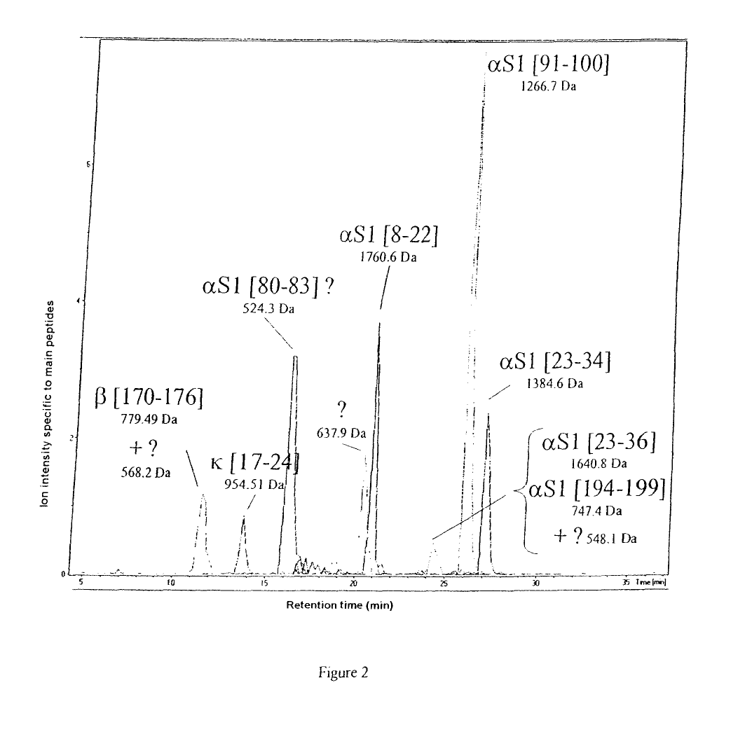

FIG. 2: Identification and quantification of the main peptides of the Lactium® ingredient by LC-MS/MS, before and after fermentation of the milk “mix” by a ferment consisting of a mixture of strains 1-2783 (CNCM deposit on 24 Jan. 2002), 1-2774 (CNAM deposit on 24 Jan. 2002), 1-2835 (CNCM deposit on 4 Apr. 2002) and 1-1968 (CNCM deposit on 14 Jan. 1998). After fermentation, these peptides are only found in trace form and merge with the base line. “?” means that identification of the sequence was not possible or is not certain; only the mass of the peptide is reported in this case.

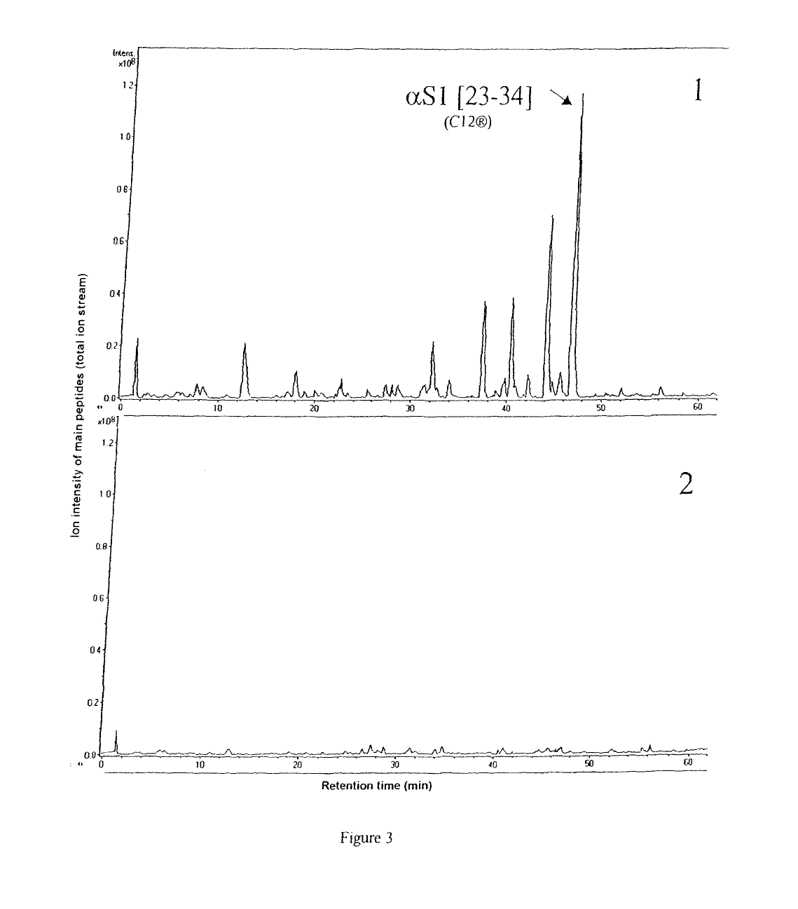

FIG. 3: Compared peptide profiles (LC-MS/MS chromatograms) of a milk “mix” containing 1.5 g/L DMV C12® hydrolysate before (1) and after (2) fermentation up to pH 4.7 by the lacetic ferment Hansen YC380. Almost all the peptides of the hydrolysate, including the bioactive C12 peptide (fragment αS1[23-34] disappeared after metabolisation by the ferment strains.

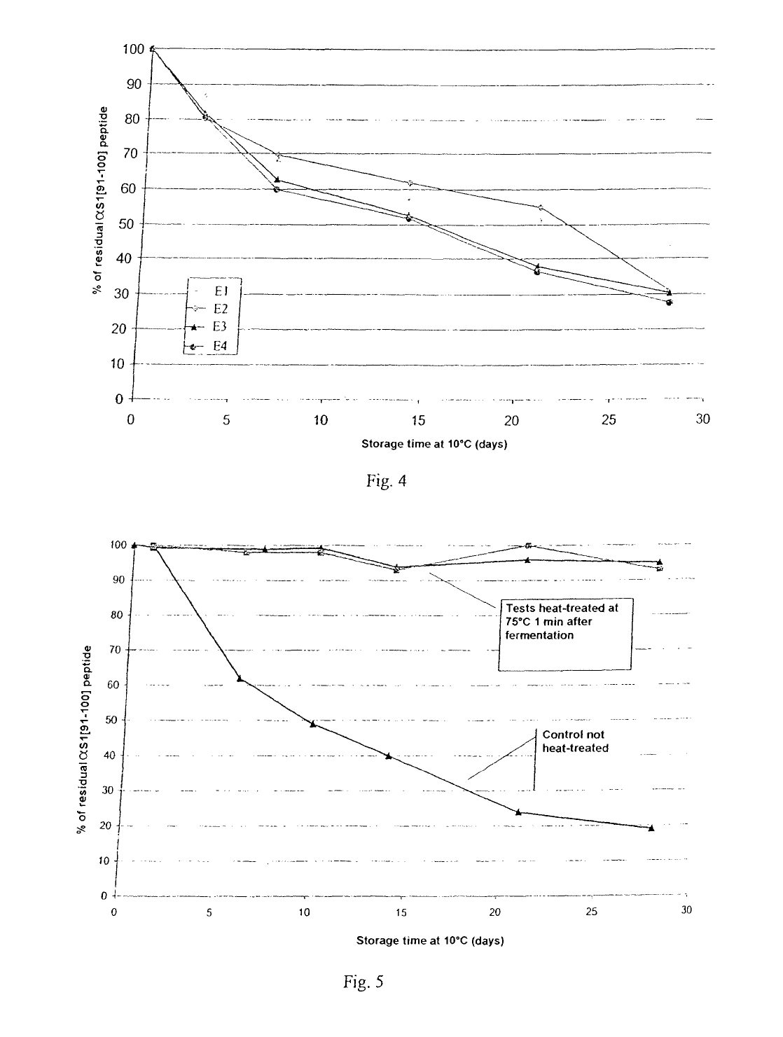

FIG. 4: Curves illustrating changes in the residual content of the αS1[91-100] bioactive peptide in an end product consisting of 95 wt. ° fermented by the ferment containing the strains 1-2783, 1-2774, 1-2835 and 1-1968, and 5° flavoured sugar syrup containing the αS1[91-100] peptide, during storage at 10° C. The experiment was performed with 4 independent tests E1, E2, E3 and E4.

FIG. 5: Curves illustrating changes in the residual content of αS1[91-100] bioactive peptide added after fermentation, in a product fermented and then thermised at 75° C. for 1 minute and stored at 10° C. until the Best Before Date (BBD).

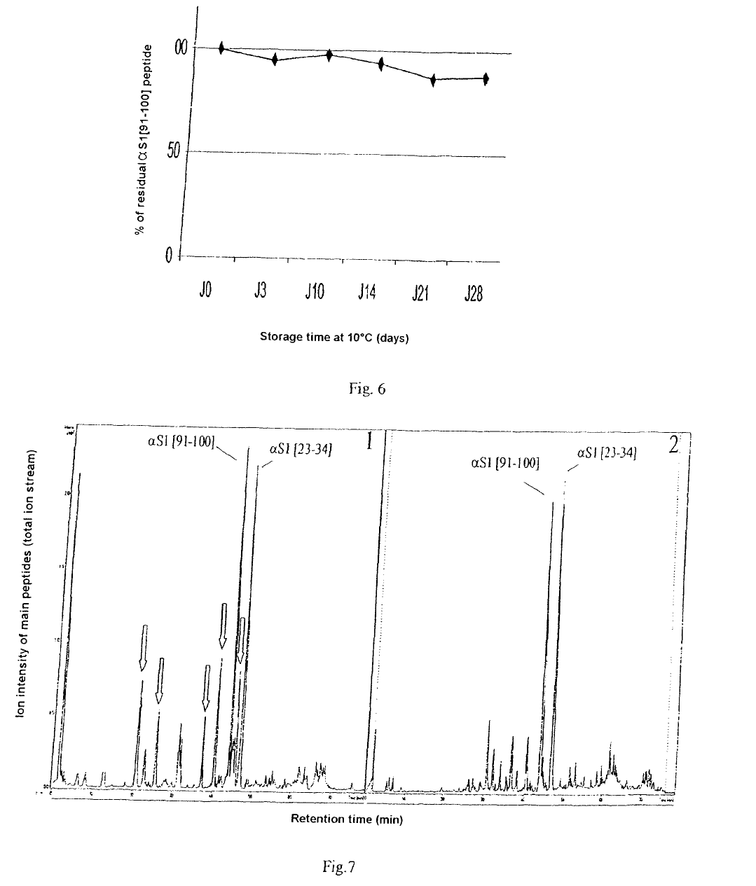

FIG. 6: Illustration of the change in content of encapsulated bioactive ingredient over time (storage at 4° C.).

FIG. 7: Compared peptide profiles (LC-MS/MS chromatograms) of a milk mix containing 1.5 g/L Lactium® added in the form of an emulsion in palm oil according to the present invention, immediately after addition to the fermented mass (1) and after 14 days' storage at 10° C. (2).

Other characteristics and advantages of the present invention will become apparent on reading the following examples given solely by way of illustration.

### EXAMPLES

### Example 1

### Use of Bioactive Ingredients of Interest without Applying the Claimed Invention

1.1 Example with the αS1[91-100] Bioactive Peptide Contained in Lactium® Hydrolysate

The use of ingredients of peptide or protein type often used in powder form, is simpler when they are added during the preparation step of the “milk” mix (milk powdering) before sanitizing heat treatment (i.e. 95° C., 8 min.) and hence before fermentation. In this case the risk of metabolising the active peptide is very high. This is the case for example when using a functional ingredient such as Lactium® (Ingredia, France) containing a bioactive peptide (fragment 91-100 of αSI casein).

Protocol: the medium was prepared by hydrating a skimmed milk powder at 120 g/L, to which 1.5 g/L of Lactium® ingredient is added (corresponding to approximately 30 mg/L αs1 [91-100] bioactive peptide) then pasteurised at 95° C. for 8 minutes.

The lacetic ferment was added at a percentage of 0.02% and fermentation was conducted at the optimal temperature of the chosen ferment (between 37 and 42° C.) up to a pH of 4.70.

Analysis of the residual peptides, in particular of the bioactive peptide αS1[91-100], was conducted using a HPLC method coupled to a detector of MS/MS type as described below:
the sample was prepared by diluting the fermented medium in a mixture of water, methanol and trifluoroacetic acid (50/50/0.1%) to a ratio of approximately 1 to 6. The supernatant after centrifuging formed the sample representing the peptide content of the fermented medium.
this sample was injected into a HPLC chromatography system of Agilent 1100 type (Agilent Technologies France, 1 rue Galvani 91745 Massy Cedex France) equipped with a column adapted for peptide analysis of Waters Symetry® type (5 μm 2.1×150 mm, WAT056975, Waters, France, 5 Rue Jacques Monod, 78280 Guyancourt) at a temperature of 40° C. and flow rate 0.25 ml/min. The peptides were eluted in conventional manner against an increasing gradient of solvent B (Acetonitrile+0.100% formic acid) in solvent A (Water+0.106% formic acid) for a time of 40 min to 2 hours in relation to desired resolution.
detection was made using a specific detector of MS/MS type, for example of ion trap type such as the Esquire 3000+(Bruker, Daltonique, rue de l'Industrie 67166 Wissembourg Cedex) adjusted either for global analysis of the peptide content (MS-MS mode) or for precise, specific quantification of a peptide from its characteristic fragments. For example the αS1[91-100] peptide was isolated from its mass (double charged ion of mass 634.5 Da) and quantified from the intensity of its characteristic daughter ions after fragmenting (ions with m/z 991.5 Da, 771.5 Da and 658.3 Da). In even more precise manner, an internal standard consisting of the same synthetic peptide deuterated twice (characteristic fragment of 993.5 Da) allowed any matrix-related interferences to be overcome and taken into account.

The results are illustrated FIG. 1

When comparing the αS1[91-100] peptide before fermentation by a ferment consisting of a mixture of strains 1-2783 (CNCM deposit on 24 Jan. 2002), 1-2774 (CNCM deposit on 24 Jan. 2002), 1-2835 (CNCM deposit on 4 Apr. 2002) and 1-1968 (CNCM deposit on 14 Jan. 1998) or by a ferment such as YC380 (Chr. Hansen S A, Le Moulin d'Aulnay, BP 64 91292 ARPAJON Cedex France), it was evidenced that more than 95% of the bioactive peptide αS1[91-100] was consumed after fermentation.

These observations show that the incorporation of bioactive peptides conforming to the above is not applicable as such, in order to obtain food products, in particular dairy products, supplemented with quantities of peptides and/or bioactive proteins that are sufficiently stable over time to observe the desired effect in consumers.

1.2 Examples with Other Bioactive Peptides of Interest

The results are illustrated in FIGS. 2 and 3.

The Lactium® ingredient contains numerous other peptides of which some have potential biological activity (such as fragment 23-34 of the αS1 casein which is also marketed in the C12 ingredient by DMV International). It is interesting to note that practically all the peptides provided by adding Lactium® are largely consumed during fermentation.

Irrespective of their origin (derived from different αS1, αS2, κ, β-caseins) and their size (2 to 3 residues up to 12 residues and more) all the peptides are globally consumed during the fermentation process.

1.3 Use of the αS1[91-100] Bioactive Peptide (Lactium® with Other Ferments

To verify that this phenomenon is not particular to the two ferments used in paragraph 1.1) above, the main industrial ferments and different pure strains entering into the composition of these ferments, were tested on the same test basis: milk reconstituted from milk powder, to which Lactium® was added at a dose of 1.5 g/L, was fermented under standard conditions (optimal ferment temperature between 37 and 42° C., halting of fermentation at pH 4.7, two repeats). Analysis of the level of αS1[91-100] bioactive peptide was conducted on the sample before and after fermentation.

The results obtained on the pure strains are given in Table 3 below:

TABLE 3

| | % αS1[91-100] peptide remaining |
|---|---|
| | after fermentation |
| Pure strains | |
| (S. thermophilus) | |
| 1-1630 (24 Oct. 1995) | 0.3 |
| 1-1477 (22 Sep. 1994) | 0.3 |
| Pure strains | |
| (Lactobacillus) | |
| 1-1632 (24 Oct. 1995) | 0.2 |
| 1-1519 (30 Dec. 1994) | 0.1 |
| 1-1968 (14 Jan. 1998) | 1.6 |
| 1-2809 (19 Feb. 2002) | 0.4 |

In Table 3 above, which reflects consumption of the bioactive peptide aS1[91-100] by different ferments and industrial strains during fermentation of a milk mix containing 1.5 g/L Lactium®, the pure strains were identified by their numbers and respective date of depositing with the CNCM (Institut Pasteur, Paris France).

Table 3 shows that all the tested ferments and strains metabolise 94 to 100% of the bioactive peptide αS1[91-100] during fermentation of a standard milk mix. The use of this ingredient is therefore impossible under conventional conditions to produce food products, in particular dairy products, containing bioactive peptides and/or proteins in quantities that are sufficiently stable over time to produce an effect in consumers.

Also, to verify that this phenomenon is not particular to the Lactium® ingredient, several combinations of ferments and other ingredients containing bioactive peptides were studied using the same test (reconstituted milk+ingredient to be tested at a dose of 1.5 g/L, fermentation under standard conditions, fermentation halted at ph 4.7, two repeats). The various combinations tested are given in Table 4 below:

TABLE 4

| | αS1[91-100] | | | |
|---|---|---|---|---|
| | peptide in | Other peptides | DMV | DMV |
| Ferments/pure strains | Lactium ® | in Lactium ® | C12 ® | CPP ® |
| Mixture of 4 strains: | | | | |
| 1-2783 (24 Jan. 2002) | | | | |
| 1-2774 (24 Jan. 2002) | X | X | X | X |
| 1-2835 (04 Apr. 2002) | | | | |
| 1-1968 (14 Jan. 1998) | | | | |
| 1-1630 (24 Oct. 1995) | X | | X | X |
| YC380 Hansen | X | X | X | X |

The ingredients C12 and CPP produced by DMV International are hydrolysates of milk proteins containing bioactive peptides respectively targeting the control of hypertension and the uptake of minerals.

In all experiments, it appeared that all the tested ferments have a large capacity to metabolise peptides irrespective of their type and size.

1.4 Addition after Fermentation

A logic alternative to the procedure tested above, is to add the functional ingredient after fermentation (process of “delayed differentiation” type), e.g. with the syrup used to flavour the fermented mass. Use of the same quantity of Lactium® ingredient according to this protocol led to the results illustrated FIG. 4.

As shown FIG. 4, even under cold addition (4° C.) after fermentation, the active peptide (provided by the equivalent of 1.5 g Lactium® per kg of end product) is rapidly degraded during storage, leaving only 30 to 40% of the initial quantity on the Best Before Date (BBD).

Therefore the population of living lacetic bacteria in the end product continues to metabolise the bioactive peptide during storage of the end product, to the extent that after only 10 days (for fresh products whose BBD is 28 days) between 35 and 50% of the peptide αS1[91-100] has disappeared, which remains unacceptable to obtain the desired effect for the consumer.

1.5 Heat Treatment of the Fermented Dairy Product Containing the Bioactive Ingredient of Interest

In this case, it is possible to ensure the stability of the αS1[91-100] peptide (FIG. 5) but to the detriment of the overall quality of the end product. This solution effectively has numerous drawbacks:
thermisation of a fermented milk mass implies the use of stabilisers added before the heat treatment (pectins, starches, carrageenans, etc.) which complicates the method and substantially increases the cost of the formula;
the industrial production line is more complex and requires specific, higher investment;
the product no longer benefits from quality labels related to products containing living ferments (yoghurt type), and thereby loses the benefits associated with the consumption of lacetic ferments;
the organoleptic impact (generally negative) is significant.

### Example 2

### Use of Bioactive Ingredients of Interest by Applying the Claimed Invention

2.1 Step 1: Encapsulation of the Bioactive Food Ingredient

Chosen encapsulating oil: palm oil (melting point=37° C.) The suppliers of this type of oil are numerous (e.g. Cargill).

The oil was melted at 50° C. to obtain total absence of crystals, and the Lactium® containing the bioactive food ingredient αS1(91-100] was gradually added under magnetic stirring (maintained at 50° C.).

2.2 Step 2: Fabrication of the Syrup-Type Aqueous Medium

A type of syrup was prepared from water and the premix of palm oil/bioactive food ingredient. The bioactive food ingredient was gradually added to the water (30° C.) under Ultraturax stirring (22000 rpm), the palm content of the solution being arbitrarily fixed at 30°.

The emulsion obtained was easily pumpable at 30° C.-35° C., but became progressively firmer as soon as the temperature fell to below 25° C.-30° C. (progressive recrystallisation of the palm oil). The use of a food emulsifier (e.g. Lactem® supplied by Danisco) to the proportion of 0.5%/fat led to obtaining a population of fatty globules that was less heterogeneous.

2.3 Step 3: Adding the Emulsion to the Fermented White Mass

As illustrated FIGS. 6 and 7, the content of bioactive ingredient is stable over time, indicating that a bioactive food ingredient can be efficiently protected by encapsulation according to the present invention.

Therefore, in addition to the αS1[91-100] peptide whose stability is shown FIG. 6, other peptides such as the αS1[23-34] bioactive peptide (present in the C12 ingredient by DMV International) are also well protected (FIG. 7). The disappearance of peptides other than those of interest (FIG. 7 (1) symbolized by arrows) accounts for some of the reduction in the global peptide content, and hence probably to the loss of bitterness in the end product.

### REFERENCES

Kayser et al., (1996) FEBS Letters 383, 18-20
Hata Y. et al., (1996) Am. J. Clin. Nutr. 64, 767-71
Nakamura Y. et al., (1995) J. Dairy Sci. 78, 1253-7
Migliore-Samour D. et al., (1988) Experimentia 44, 188-93
Defilippi C. et al., (1995) Nutr. 11, 751-4
Tomé D. et al., (1987) Am. J. Physiol. 253, G737-44
Tomé D. et al., (1988) Reprod. Nutri. Devélop. 28, 909-18
Ben Mansour A. et al., (1988) Pediatr. Res. 24, 751-5
Mahé S. et al., (1989) Reprod. Nutri. Dévelop. 29, 725-32
Schusdziarra V. et al., (1983) Diabetologia 24, 113-6
Yvon M. et al., (1994) Reprod. Nutri. Dévelop. 34, 527-37
Zucht H. D., et al., (1995) FEBS Letters 372, 185-8
Tomita M. et al., (1994) Acta Paed. Jap. 36, 585-91
Lahov E. et al., (1996) Food Chem. Toxic. 34, 131-145
Migliore-Samour D. et al., (1989) Int. Dairy Res. 56, 357-62
Jollès P. et al., (1986) Europ. J. Biochem. 158, 379-82
Raha S. et al., (1988) Blood 772, 172-8
Chabance B. et al., (1995) Brit. J. Nut. 73, 582-90
Kohmura M. et al., (1989) Agric. Biol. Chem. 53, 2107-14
Masuda O. et al., (1996) J. Nutr. 126, 3063-8
Yamamoto N. et al., (1994) Biosci. Biotech. Biochem. 58, 776-8
Ermisch A. et al., (1983) J. Neurochem. 41, 1229
Umbach M. et al., (1985) Regul. Pept. 12, 223-30
Singh M., et al., (1989) Pediatr. Res. 26, 34-8
Svedberg J. et al., (1985) Peptides 6, 825-30
Teschemacher H., et al., (1986) J. Dairy Res. 53, 135-8
Yoshikawa M. et al., (1986) Agric. Biol. Chem. 50, 2419-21
Chiba H. et al., (1989) J. Dairy Sci. 72, 363
Beucher S. et al., (1994) J. Nutr. Biochem. 5, 578-84
Parker F. et al., (1984) Eur. J. Biochem. 45, 677-82
Otani H. et al., (1992) Milchwiss. 47, 512-5
Otani H. et al., (1995) J. Dairy Res. 62, 339-48
Drouet et al., (1990) Nouv. Rev. Fr. Hermatol. 32, 59-62
Mullaly M. et al., (1997) Int. Dairy J. 7, 299-303
Meisel H. et al., (1986) FEBS Letters 196, 223-7
Danone World Newsletter N°17 (September 1998)
Functional Food Science in Europe (1998) British Journal of Nutrition 80 (1): S1-S193
Directional Paired Comparison Method, Sensory Evaluation of Food, Harry T. Lawless, Hildegarde Heymann (1990)

1. Food product containing one or more living microorganisms and at least one bioactive food ingredient of interest, characterized in that said bioactive food ingredient(s) of interest are protected physically by encapsulation in a fat, so that their metabolisation by said living microorganism(s) is reduced.

2. Food product according to claim 1, characterized in that the residual quantity of bioactive food ingredient(s) of interest in said food product, 3 weeks after its preparation, is between approximately 50 and 100% compared with the quantity of bioactive food ingredient(s) of interest present in the product just after its preparation.

3. Food product according to claim 2, characterized in that said residual quantity lies between approximately 80 and 100% compared with said quantity of bioactive food ingredient(s) of interest present in the product just after its preparation.

4. Food product according to any of claims 1 to 3, characterized in that said bioactive food ingredient(s) of interest are chosen from among:
proteins,
peptides,
vitamins,
micronutrients,
analogs or derivatives thereof, and
their combinations

5. Food product according to claim 4, characterized in that said bioactive food ingredient(s) of interest are chosen from among:
proteins,
peptides,
analogs or derivatives thereof, and
their combinations.

6. Food product according to claim 4 or 5, characterized in that said bioactive food ingredient(s) of interest are chosen from among: the αS1[91-100] peptide, C6-αₛ₁194-199 peptide, C7-β177-183 peptide, C12-αₛ₁23-34 peptide, the caseinophosphopeptides, α-casomorphin, α-casein-exorphin, casokinin, β-casomorphin, the caseinomacropeptides and glyco-macropeptides, casoxin, casoplatelins, fragments 50-53, β-lactorphins, lactoferroxin, the peptides Val-Pro-Pro, Lys-Val-Leu-Pro-Val-Pro-Gln, Tyr-Lys-Val-Pro-Gln-Leu, Tyr-Pro, Ile-Pro-Pro, fragments, analogs, derivatives thereof, proteins and/or peptides containing the same, and their combinations.

7. Food product according to any of claims 1 to 6, characterized in that it has reduced bitterness.

8. Food product according to any of claims 1 to 7, characterized in that said fat is chosen from among animal fats, in particular milk or fish, and vegetable fats.

9. Food product according to claim 8, characterized in that said vegetable fat is chosen from among palm oil, rapeseed oil and an alga-extracted fat.

10. Food product according to any of claims 1 to 9, characterized in that said living microorganism(s) have an intact or reduced capacity to metabolise said bioactive food ingredient(s) of interest.

11. Food product according to any of claims 1 to 10, characterized in that said living microorganism(s) are living bacteria, preferably living lactic bacteria.

12. Food product according to claim 11, characterized in that said living bacteria are chosen from among:
Streptococcus spp, preferably Streptococcus thermophilus
Lactobacillus spp;
Lactococcus spp;
Bifidobacterium spp.

13. Food product according to claim 12, characterized in that it contains at least the living bacteria S. thermophilus and Lactobacillus spp.

14. Food product according to claim 12 or 13, characterized in that said living S. thermophilus bacteria are chosen from among:
Streptococcus thermophilus deposited with the CNCM collection on 24 Jan. 2 under number 1-2774;
Streptococcus thermophilus deposited with the CNCM collection on 24 Oct. 1995 under number 1-1630;
Streptococcus thermophilus deposited with the CNCM collection on 10 May 4 under number 1-3211;
Streptococcus thermophilus deposited with the CNCM collection on 16 Sep. 2004 under number 1-3301.
Streptococcus thermophilus deposited with the CNCM collection on 16 Sep. 2004 under number 1-3302.

15. Food product according to claim 14, characterized in that said living bacteria are S. thermophilus bacteria deposited with the CNCM collection on 10 May 4 under number 1-3211.

16. Food product according to any of claims 1 to 15, characterized in that it also contains at least one decoy food ingredient.

17. Food product according to any of claims 1 to 16, characterized in that it is a beverage, preferably a water.

18. Food product according to any of claims 1 to 17, characterized in that it is a fermented product.

19. Food product according to any of claims 1 to 18, characterized in that it is a dairy or plant product.

20. Method for encapsulating at least one bioactive food ingredient of interest in a fat, at least comprising:
a) preparing a water/fat mixture, maintained at a temperature close to the melting point of said fat, preferably within a range of ±10° around said melting point; and
b) incorporating the bioactive ingredient of interest, progressively and under stirring, into the mixture obtained at step a), maintaining the whole at a temperature close to the melting point of said fat, preferably within a range of ±10° around said melting point.

21. Method according to claim 20, also comprising the addition of one or more food additives such as emulsifiers, thickeners, etc. and mixing the whole while maintaining a temperature close to the melting point of said fat, preferably within a range of ±10° around said melting point.

22. Method for preparing a food product according to any of claims 1 to 19, characterized in that said bioactive food ingredient(s) of interest are encapsulated before being added to the mixture intended to form said food product.

23. Method according to claim 22, characterized in that said bioactive food ingredient(s) of interest are encapsulated according to the method in claim 19 or 20.

24. Method according to claim 22 or 23, characterized in that said encapsulated bioactive food ingredient(s) of interest are added to said mixture under stirring.

25. Method according to any of claims 22 to 24, characterized in that:
said food product is fermented; and
said encapsulated bioactive food ingredient(s) of interest are added to said mixture before or after, preferably after, fermentation.

26. Use of a food product according to any of claims 1 to 19 as a functional food.

27. Use of a fat to encapsulate at least one bioactive food ingredient of interest intended to be incorporated in a food product containing one or more living microorganisms.
